(19) United States
(12) Patent Application Publication
(10) Pub. No.: US 2014/0296611 A1
(43) Pub. Date: Oct. 2, 2014

(54) Brachytherapy Radiation Protective Shield and Method for Protecting Against Radiation

(71) Applicant: David Schwartz, Brookville, NY (US)

(72) Inventor: David Schwartz, Brookville, NY (US)

(21) Appl. No.: 13/873,475

(22) Filed: Apr. 30, 2013

Related U.S. Application Data
(60) Provisional application No. 61/805,455, filed on Mar. 26, 2013.

(51) Int. Cl. A61N 5/10

(52) U.S. Cl. CPC A61N 5/1007; A61N 2005/1094
USPC 600/8; 600/7

(57) ABSTRACT

A lightweight, portable, interchangeable and effective shield that will protect from radiation during the radioactive seed insertion and positioning in brachytherapy. The shield is attached to the matrix used to position and guide the needles with the radioactive seeds. The shield minimizes the radiation exposure as much as reasonably possible because any amount of radiation exposure carries a risk to the exposed individual.

### CROSS REFERENCE OF RELATED APPLICATION

This patent application is a nonprovisional application of provisional patent application Ser. No. 61/805,455 filed on Mar. 26, 2013.

### BACKGROUND

This invention relates to a radiation protective shield and method of use thereof for protecting the physician, who is typically a radiation oncologist or a surgeon, during the cancer treatments of patients.

Brachytherapy is short-distance internal radiotherapy, in which small radioactive seeds or pellets are implanted into tumors of cancerous tissue and kill cancerous cells. Brachytherapy is often an alternative to the radically-invasive surgical tumor removal, or external beam radiation. Brachytherapy is a more localized and targeted treatment, and it has been successfully used to treat various cancers, including prostate cancer that is used as an example to describe the present invention.

Prostate cancer is the second leading cause of cancer death in men, and this cancer accounts for approximately a third of all male cancers. Early diagnosis and treatment of the disease significantly improves survival chances and minimizes harmful side effects from treatment. Prostate biopsies are a common way to diagnose the cancer in a male patient's prostate. The prostate biopsies are typically performed through the rectum and are called in the medical field “a transrectal biopsy.” An alternative way to perform prostate biopsies is transperineally, or through the perineum, which is the tissue between the testicles and the anus of a male patient.

In prostate brachytherapy, patients are placed in the lithotomy position, where the patient is supine on his back, and the patient's legs are elevated and/or bent. The lithotomy position allows for unobstructed access to the perineum.

With an ultrasound probe in the rectum looking at the prostate just anterior to the rectum, needles are placed through the skin of the perineum into the prostate. A template grid that corresponds to an associated grid on the ultrasound screen is utilized to guide the needles. Other guiding methods, such as x-ray and fluoroscopy, have also been used. The template grid is typically a square matrix with evenly-spaced apertures for inserting the needles, as illustrated in FIG. 3, and each of the apertures is associated with a horizontal and a vertical coordinates.

In prostate brachytherapy, which uses radiation sources placed in or near the area to be treated, small radioactive seeds or pellets are implanted into cancerous tissue in the prostate through needles. The needles are guided by the matrix through the perineum and into the prostate to properly position and implant the seeds. This is also called permanent brachytherapy or seed implantation. The template grid and the insertion of the needles guided by ultrasound allow for precise positioning of the radioactive seeds inside the cancerous tumor tissue.

When implanted, the radioactive seeds remain lodged in the cancerous tissue and continue delivering a localized dose of radiation directly to the cancerous tissue.

However, the radioactive seeds inserted into and positioned inside the prostate expose treating physicians to radiation, which, even in small amounts, is highly undesirable if the exposure is prolonged from patient to patient. The radioactive seeds typically used are Iodine-125, Palladium-103 and Cesium-131, with the radiation emitted from 04 to 2.5 u per seed, with average energy of 21 keV for PD-103, 29.5 keV for 1-125, and 30.4 keV for Cs-131. However, dozens or sometimes over a hundred seeds are implanted into any given patient, and the radiation exposure is multiplied by the number of patients treated every day.

### DESCRIPTION OF PRIOR ART

The current state of the art involves using a simple square matrix, which offers little or no protection against the radiation emitted by the radioactive seeds. The insertion and positioning of the radioactive seeds and the removal of the needles is performed without any portable shielding that would protect the physician during the procedure.

What is needed is a lightweight, portable, and efficient shield that will protect the physician from radiation during the procedure. Any radiation exposure, no matter how small, carries some risk to the exposed individual. There is a known concept of “ALARA”, which stands for “As Low As Reasonably Achievable” and is related to minimizing the exposure associated with use of radiation or radioactive materials, especially exposure that is unnecessary and could be avoided. ALARA utilizes shielding, whenever possible, as one of the basic protective measures used to minimize the radiation exposure, together with reducing the time of the exposure, and maximizing the distance from the radiation source.

### SUMMARY

This invention meets the current need for a radiation shield. A novel radiation protective shield and a method for use thereof are provided. The preferred embodiment of the radiation shield is a substantially flat protective plate that attaches to the matrix and shields the user from radiation. The shield follows and fits with the ALARA concept, minimizing the radiation exposure associated with the handling and use of the radioactive seeds.

The shield is preferably removably attached to the matrix for ease of cleaning and replacement, but the shield can also be permanently attached to the matrix or even formed with the matrix as a one-piece unit. The preferred method of attachment of the shield to the matrix uses two spring clips as illustrated in FIG. 1.

### BRIEF DESCRIPTION OF THE DRAWINGS

These features, aspects and advantages of the novel radiation protective shield and a method of use thereof will become further understood with reference to the following description and accompanying drawings where

FIG. 1 is an isometric view of the matrix and the attached protective shield of the present invention;

FIG. 2 is a front down view of the matrix and the attached protective shield of the present invention illustrated in FIG. 1; and

FIG. 3 is a close-up view of the matrix (the template grid).

### DESCRIPTION

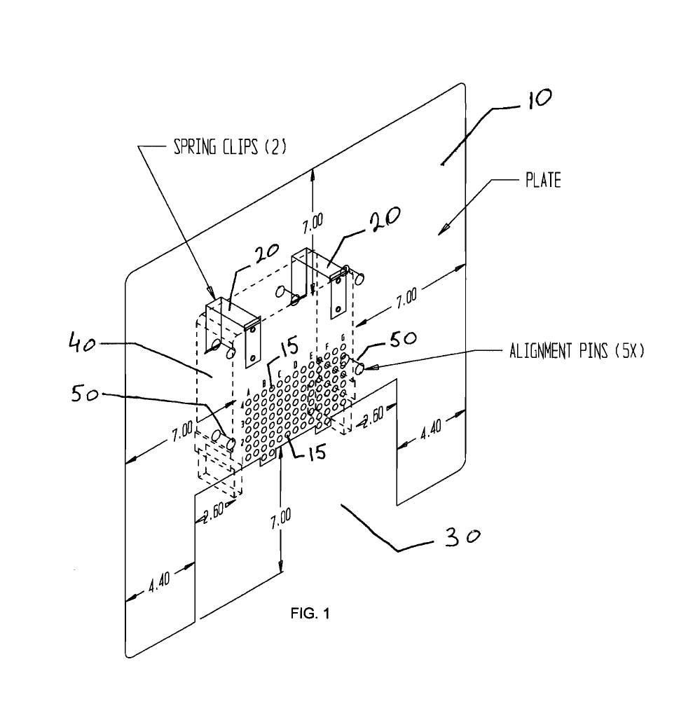

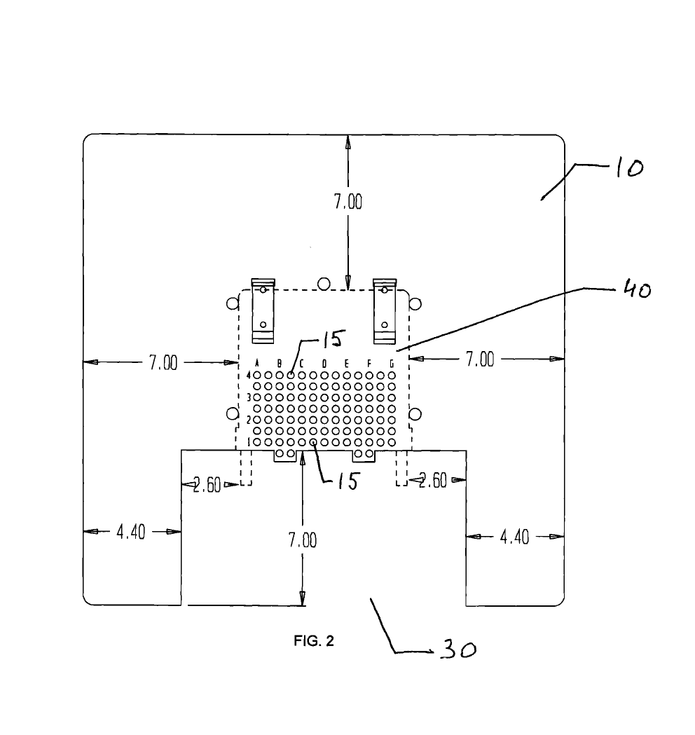

The present invention is directed to an effective, lightweight, portable radiation protective shield plate and a method of use thereof. A preferred embodiment is shown in FIGS. 1 and 2. With reference to the drawings, and particularly FIGS. 1 and 2, one embodiment of the invention comprises a shield plate 10 removably attached to the matrix 40 using two spring clips 20 fastened to the shield, which clips 20 snap onto the matrix 40 over the top surface of the matrix 40.

However, other types and numbers of clips or brackets may be used to attach the shield plate 10 to the matrix 40 and to hold it in place, such as C-shaped brackets for example. The clips or brackets may snap in or otherwise securely fasten the shield plate 10 to the matrix 40. It is also possible to use railed attachment methods, or screws or bolts (not shown) for a more permanent and stable coupling of the shield plate 10 and the matrix 40.

The shield plate 10 is preferably portable, capable of being lifted and manipulated without strain by an average user. However, the size of the shield plate 10 may be varied based on the need dictated by the application of the radioactive seeds and a variety of other factors. Therefore, it is preferable to have a shield plate 10 that is easily removable, i.e., by detaching or disengaging the clips 20 (or clamps, brackets or other attachment means), so that a number of interchangeable plates of different size, material and thickness may be used with the matrix 40.

The shield plate 10, when attached to the matrix 40 and is in use, is preferably positioned between the matrix 40 and the treating physician. The shield plate 10 extends beyond the edges of the matrix 40, preferably for the distance approximately equal to the width of the matrix 40 on each side. However, larger or smaller shields may be employed as necessary. In the shown preferred embodiment, there is a cutout 30 in the shield plate 10 below the bottom edge of the matrix 40. The cutout 30 is necessary to avoid other equipment used in the procedure.

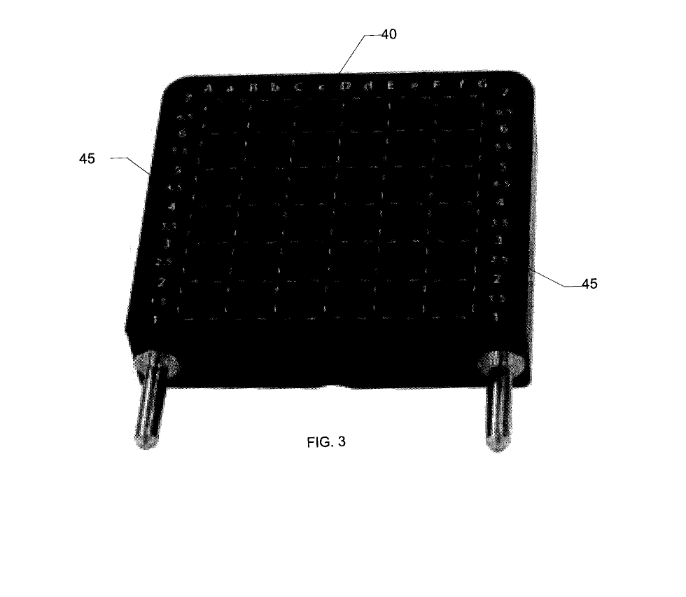

When the shield plate 10 covers the entire matrix 40, which is the preferred configuration, the shield plate 10 has apertures 15 corresponding to the apertures 45 on the matrix 40, illustrated in FIG. 3, to allow the needles with the radioactive seeds to pass through the shield plate 10 and the matrix 40. However, in another embodiment (not shown), the shield plate 10 does not fully overlap the area of the matrix 40 with the apertures 45, so the cutout 30 in the shield plate 10 extends above the edge of the lower surface of the matrix 40 and terminates just above the upper row of the apertures 45.

When in use, the present invention shields the user during the brachytherapy procedure. More specifically, the protective shield plate 10 of the present invention protects the user from radiation during the insertion and positioning of the radioactive seeds, while the user inserts the needles with the radioactive seeds through the apertures 15 of the shield plate 10 and the apertures 45 of the matrix 40, punctures the skin in the perineum area with the needles and implants the seeds into the prostate, where the radioactive seeds are positioned using the vertical and horizontal coordinates associated with the apertures 45 on the matrix 40, ultrasound imaging or similar guidance, and the length of the needles.

To facilitate easier attachment of the protective shield plate 10 to the matrix 40, as well as the proper, symmetric alignment of the shield plate 10 with the matrix 40, the shield plate 10 preferably has alignment pins 50 as illustrated in FIG. 1, which are preferably structures attached to the posterior surface of the shield plate 10 that hold the matrix 40, also known as the template or grid, in place.

The preferred embodiment uses alignment pins 50 attached to the posterior of the shield plate 10 only, and, as illustrated in FIGS. 1 and 2, there are five alignment pins 50 in the preferred embodiment that are positioned around the side edges and the top edge of the matrix 40. The alignment pins 50 are essentially pegs that may be formed with the shield plate 10 or formed separately and affixed to the posterior of the shield plate 10 (for example riveted). The placement of the alignment pins 50 allows for the proper alignment of the shield plate 10 with the matrix 40, so as to ensure the alignment of the apertures 45 in the matrix and the apertures 15 in the shield plate 10 when the shield plate 10 covers the entire matrix 40. This attachment method and location of the alignment pins 50 also allow for the use of the protective shield 10 of the present invention with any standard size matrix.

The shield plate 10 is preferably made from stainless steel. Various other materials with radiation shielding properties may be used for the shield plate 10, such as leaded glass or acrylic glass used to protect against X-Ray exposure, as well as numerous other materials and compounds. Other embodiments of the present invention may use an acrylic shield plate 10 due to its light weight and resistance to shattering. The corners and the corner edges of the shield plate 10 are preferably rounded and/or smoothed so as to avoid any cut or other injury to the patient or the user.

The protective radiation shield 10 and method of use thereof of the present invention have other possible uses. Although the present invention has been described in terms of use for prostate cancer treatment, brachytherapy has been successfully used for breast, cervical, lung, skin, head, and neck cancers, and the present invention can be applied to all of these treatments. Likewise, it may be used for contact brachytherapy where the radiation sources are placed outside of, but next to, the targeted tumor, as well as internal brachytherapy.

The above description of the disclosed preferred embodiments is provided to enable any person skilled in the art to make or use the invention. Various modifications to these embodiments will be readily apparent to those skilled in the art, and the principles described herein can be applied to other embodiments without departing from the spirit or scope of the invention and the subject matter of the present invention, which is broadly contemplated by the Applicant. The scope of the present invention fully encompasses other embodiments that may be or become obvious to those skilled in the art.

1. A novel radiation shield for protecting a user against radiation during brachytherapy treatment of a patient utilizing a matrix, the radiation shield comprising:
(a) a substantially flat protective plate attached to the matrix, said substantially flat protective plate shielding the user from radiation.

2. The novel radiation shield of claim 1, wherein the substantially flat protective plate is removably attached to the matrix.

3. The novel radiation shield of claim 1, wherein the substantially flat protective plate is permanently attached to the matrix.

4. The novel radiation shield of claim 1, wherein the substantially flat protective plate and the matrix include a plurality of cooperating apertures suitable for brachytherapy needles, each of the plurality of cooperating apertures having associated vertical and horizontal coordinates.

5. The novel radiation shield of claim 2, wherein the substantially flat protective plate is removably attached to the matrix by at least one spring clip.

6. The novel radiation shield of claim 5, wherein the at least one spring clip couples with a top surface of the matrix to removably attach the substantially flat protective plate to the matrix.

7. The novel radiation shield of claim 1, wherein the substantially flat protective plate includes a cutout extending to a bottom surface of the matrix.

8. The novel radiation shield of claim 1, wherein the substantially flat plate includes a plurality of alignment pins to facilitate the alignment of the substantially flat plate and the matrix.

9. The novel radiation shield of claim 8, wherein each of the plurality of alignment pins is a structure attached to a surface of the substantially flat plate.

10. The novel radiation shield of claim 1, wherein the substantially flat plate is portable, capable of being lifted and manipulated by an average user.

11. The novel radiation shield of claim 1, wherein corners of the substantially flat plate are rounded so as to avoid injury to the patient or the user.

12. The novel radiation shield of claim 1, wherein edges of the substantially flat plate are smoothed so as to avoid injury to the patient or the user.

13. A novel radiation shield for protecting a user against radiation during brachytherapy treatment of a patient utilizing a matrix, the radiation shield comprising:
(a) a substantially flat protective plate having at least one fastener for removably attaching the substantially flat protective plate to the matrix, said substantially flat protective plate shielding the user from radiation.

14. The novel radiation shield of claim 13, wherein the at least one fastener is selected from the group consisting of clips, spring clips, clamps, brackets, magnets, Velcro, reciprocating rails, screws, bolts and combination thereof.

15. A novel method of protecting a user against radiation during brachytherapy treatment of a patient, comprising:
(a) attaching a substantially flat protective plate to a matrix, said substantially flat protective plate and said matrix having a plurality of cooperating apertures suitable for brachytherapy needles, wherein each of the plurality of cooperating apertures is associated with a vertical coordinate and a horizontal coordinate;
(b) positioning the substantially flat protective plate between the matrix and the user, wherein the matrix is positioned between the patient and the substantially flat protective plate and wherein the substantially flat protective plate protects the user from radiation;
(c) inserting at least one brachytherapy needle having at least one radioactive seed therein through one of the plurality of cooperating apertures of the substantially flat protective plate and the matrix;
(d) puncturing skin of the patient near a region of cancerous tissue with the at least one brachytherapy needle; and
(e) positioning the at least one radioactive seed inside the cancerous tissue.

16. The method of claim 15, wherein the positioning of the radioactive seeds is performed using the vertical coordinate and the horizontal coordinate associated with each of the plurality of cooperating apertures on the substantially flat protective plate and the matrix.

17. The method of claim 15, wherein the positioning of the at least one radioactive seed is performed using ultrasound imaging.

18. The method of claim 15, wherein the positioning of the at least one radioactive seed is performed using the length of the at least one brachytherapy needle.
